(19) United States
(12) Patent Application Publication
(10) Pub. No.: US 2021/0183501 A1
(43) Pub. Date: Jun. 17, 2021

(54) Systems and Methods for Ensuring Facility Compliance

(71) Applicant: Kimberly Geis, Napa, CA (US)

(72) Inventor: Kimberly Geis, Napa, CA (US)

(21) Appl. No.: 16/664,807

(22) Filed: Oct. 26, 2019

(51) Int. Cl. G16H 40/20; G06Q 30/00; G06Q 10/06; G16H 10/60; G16H 15/00; G06Q 50/26; G16H 20/10; A61J 7/04

(52) U.S. Cl. CPC G16H 40/20; G06Q 30/018; G06Q 10/0635; G16H 10/60; A61J 2200/30; G06Q 50/26; G16H 20/10; A61J 7/04; G16H 15/00

(57) ABSTRACT

An electronic record system and method to operate a state licensed facility. The records system of the present invention meets the daily needs of the administrator and staff caring for the resident in a residential care setting. Services provided by staff include activities of daily living and medication administration for residents. As staff document a care note the records system transfers this data to a central timeline for that resident. This timeline shows a user all medications given as well as ADL's, IADL's and care notes. The present invention will streamline all required documents for the Facility, Staff and Resident. This invention digitalizes the process of operating a State licensed Residential Care Facility for the Elderly (RCFE) and Adult Residential Facility (ARF). This invention helps the facility owner maintain compliance allowing more efficiently in are for the residents. This invention and method allow for Regulatory and licensing agency to access and verify document compliance for each facility. This method and process allows for facility owners and operators to access required documents in the event originals are missing or damaged during disaster or for immediate need to review.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

This invention relates to systems and methods to ensure facility compliance. More specifically, the present invention creates an electronic record system to operate a State licensed facility also known as Assisted Living Facility, Adult Residential Care Facility for the Elderly, Board and Care Home, Adult Residential Facility.

### Description of the Related Art

In California state, As of Jun. 30, 2017 Community Care Licensing Division has licensed approximately 72,400 facilities with the total capacity to serve 1.4 million Californians. Of these facilities 7900 are residential Care facilities for the Elderly with a capacity to serve 200,000 elders in need of care. Of these 7900 RCFE's 5500 are 6 beds and fewer. These Facility services are unique and non-medical paid for with private funds from residents.

Currently all required documents used to operate a state licensed facility are printed and hand signed. These documents are stored on site and not accessible. By using the present invention, all required documents will be accessible from anywhere with an Internet connection using a digital device such as a computer, phone or tablet. With the present invention, the facility administrator, licensee and State licensing agents can perform compliance reviews on facilities from their office locations. Digitizing the facility saves time and money. It gives the facility administrator, licensee and State licensing agents the ability to perform compliance audits on documents from a distance to ensure better resident care and compliance with state laws.

There are several non state specific assisted living software programs, they are very complex and contain terminology associated with medical services whereas these facilities are providing non-medical facilities. These programs are difficult to use and can take a user many hours of valuable time to learn, they are very overwhelming covering requirements typical to the home health care industry including Doctors, Nurses, Therapists. Six-bed facility owners do not have time to learn and teach a complex program with all sorts of features but instead need a tool to help keep them in compliance. The present invention is a system and method that focuses on a filing system for all required documents, it is a simple use allows staff to easily chart. Activities of Daily Living (ADL's), Instrumental activities of daily living (IADL's), care notes and medication passes. This documentation allows for ensuring quality of care.

Other Facilities that can use this invention systems and methods for compliance include Home care organizations, child care centers, infant centers, school age centers, childcare centers, family care home, group homes, small family homes and foster family home

### BRIEF SUMMARY OF THE INVENTION

In one aspect, the systems and method of the present invention for ensuring facility document compliance

In another aspect, the present invention relates to systems and methods for ensuring facility document compliance allowing all required documents of the state licensed facility to be accessible from anywhere with an internet connection using a digital device such as a computer, phone or tablet.

In yet another aspect, the system and method of the present invention let the states can perform compliance reviews on facilities from their office locations.

In yet another aspect, the system and method of the present invention are to digitize the facility to save time and money, data entered for each profile feeds to the required document which can be printed, emailed and digitally signed.

In yet another aspect, the system and method of the present invention gives the state regulatory agency the ability to perform audits on documents and compliance from a distance to ensure better resident care and compliance with the state laws.

In yet another aspect, the system and method of the present invention focused on a filing system for all caregiver facilities required documents.

In yet another aspect, the system and method of the present invention are simple to use and allow staff to easily chart IADLs and ADL's, care notes, medications passed so they can spend more time caring for their residents.

In yet another aspect, the system and method of the present invention are to Improve the quality of care services in facilities.

In yet another aspect, the system and method of the present invention is to reduce caregiver errors in facilities.

In yet another aspect, the system and method of the present invention is to reduce medication errors in facilities

In yet another aspect, the system and method of the present invention is to allow licensees and administrators to run and operate multiple state licensed facilities from anywhere using internet.

In yet another aspect, the system and method of the present invention provides a secure central location to store all facility documentation.

In yet another aspect, the system and method of the present invention provides tracking of the caregiver's activities, the status of the resident's medications and the overall status of the facilities documentation.

In yet another aspect, the system and method of the present invention collates all the resident's information together to provide an overview of the resident's care—basic information, medical information, medication records, care note and ADL records, etc.

In yet another aspect, the system and method of the present invention allows for standard and custom reporting for facilities, LPA's and state agencies.

### DESCRIPTION OF THE DRAWINGS

Having thus described the disclosure in general terms, reference will now be made to the accompanying figures, wherein:

FIG. 1. Illustrates the modules of the record system software.

FIG. 2. Illustrates the administrator dashboard module.

FIG. 3. Illustrates the resident module.

FIG. 4. Illustrates the medications module.

FIG. 5. Illustrates the caregivers module.

FIG. 6. Illustrates the staff module.

FIG. 7. Illustrates the facility module.

FIG. 8. Illustrates the admin module.

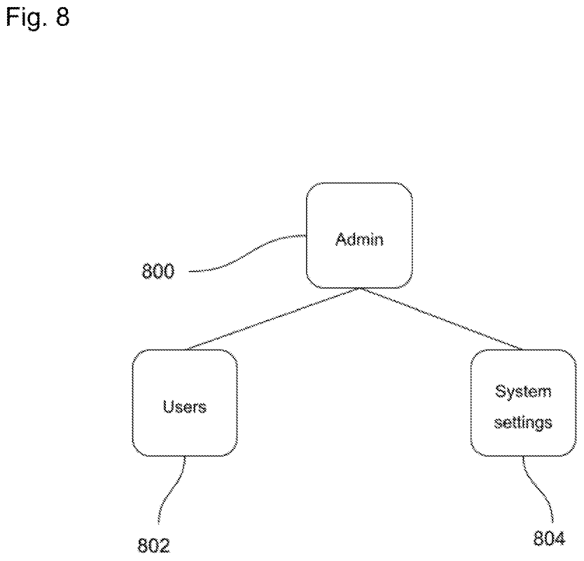

FIG. 9. Illustrates Licensed Program Analyst (LPA).

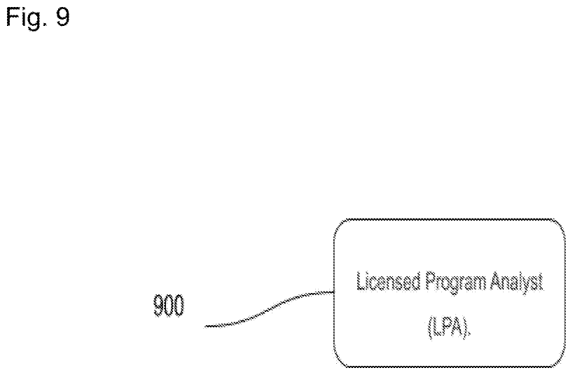

It should be noted that the accompanying figure are intended to present illustrations of exemplary embodiments of the present disclosure. This figure is not intended to limit the scope of the present disclosure. It should also be noted that accompanying figure is not necessarily drawn to scale.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

Reference will now be made in detail to selected embodiments of the present disclosure in conjunction with accompanying figures. The embodiments described herein are not intended to limit the scope of the disclosure, and the present disclosure should not be construed as limited to the embodiments described. This disclosure may be embodied in different forms without departing from the scope and spirit of the disclosure. It should be understood that the accompanying figures are intended and provided to illustrate embodiments of the disclosure described below and are not necessarily drawn to scale. In the drawings, like numbers refer to like elements throughout, and thicknesses and dimensions of some components may be exaggerated for providing better clarity and ease of understanding.

It should be noted that the terms “first”, “second”, and the like, herein do not denote any order, ranking, quantity, or importance, but rather are used to distinguish one element from another. Further, the terms “a” and “an” herein do not denote a limitation of quantity, but rather denote the presence of at least one of the referenced items.

All illustrations of the drawings are for the purpose of describing selected versions of the present invention and are not intended to limit the scope of the present invention.

The records system software of the present invention meets the daily needs of the administrator and staff caring for residents. This records system software keeps up to the minute at fingertips information. The records system of the present invention based on a monthly subscription. The monthly subscription of the record system software will streamline all required documents for the Residential Care Facility for the Elderly (RCFE), this includes all the documents needed for staff, resident, facility and medications. The records system software digitalizes the process of operating a California State licensed facility such as RCFE and ARF. This records system software helps the facility owner remain in compliance with all CCLD DSS (Community Care Licensing Division Department of Social Services) title regulations.

The records system software of the present invention will be targeting all State licensed Facilities in California, there are approximately 7500 licensed facilities in California with 500 pending licensing statuses. There are 5500-6 bed facilities targeted that have not converted to an electronic facility record, It is hard for them to operate as the owner/operator must do assessments, discharges, hospice care, prescription calling and pick up working shifts caring for elderly. Every facility has an administrator, this administrator is responsible for all facility, staff, resident and medication documents and for a 6-bed facility it over 133 required documents.

The user will upload all required document forms and the program has default expiration dates, as a form becomes expired and requires review, an alert will be sent to dashboard. The program focuses on residents, staff and facility required documents as well as medication pass and pour. Beside the records system software of the invention, there is a process where the user fills out all questions and uploads all required documents into the system. The program is made up of coding that runs the program.

The software of the present invention is designed for both the individual care homes and the compliance agencies. The Software allows the RCFE's portal to manage the numerous documents that they are required to maintain to remain in good standing with the State of California as well as assist them with day-to-day operations.

The software is a secure website that was custom built using industry leading technologies for flexibility and security with a focus on a simple user-friendly interface.

The software is secure to handle both HIPPA and PII (Personal Identifying Information, see CA bill number SB 1386) data. This system also adds the benefit that all the documentation will be stored electronically providing business and records continuity in the event of a fire or other disaster.

The software of the present invention provides facility information stored electronically in the system and can be viewed by the care home owner, administrator, or state officials from any location using a secure login.

By using the present invention software, the facility compliance is automatically analyzed and documented based on the care home's classifications. The items that are out of compliance are highlighted on the administrator's dashboard. The administrator can then work to resolve the compliance issue(s).

Resident information is readily available to the staff in the facility. The system will keep track of everything related to each resident and provides a recap of their care for inquiries from family, doctors, etc.

To use the records system software of the present invention, a user will sign up, then the user will go to the payment page to pay the fee of 69.00 per month through paypal button then receive welcome email within 24 hrs.

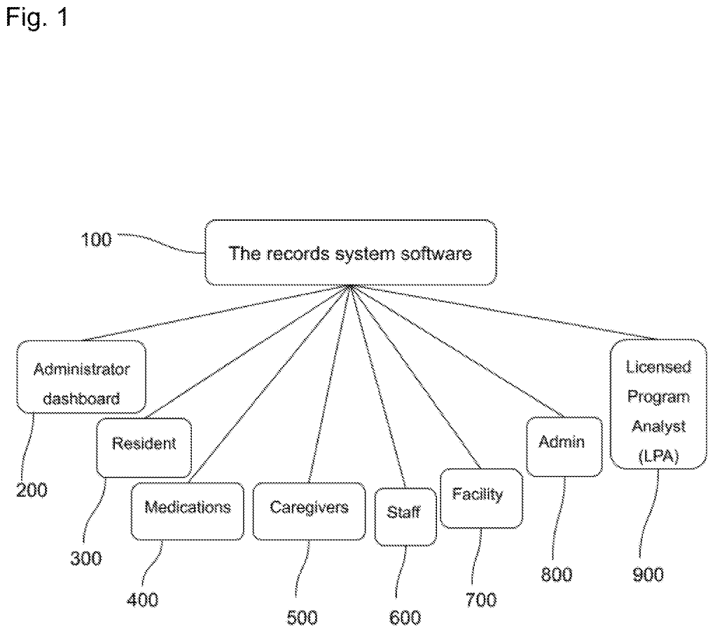

Referring to FIG. 1, after a user login, the user will see the modules of administrator dashboard 200, resident 300, medications 400, caregivers 500, staff 600, facility 700, admin 800 and Licensed Program Analyst (LPA) 900.

In the following parts we will explain every module in details:

### Administrator Dashboard Module

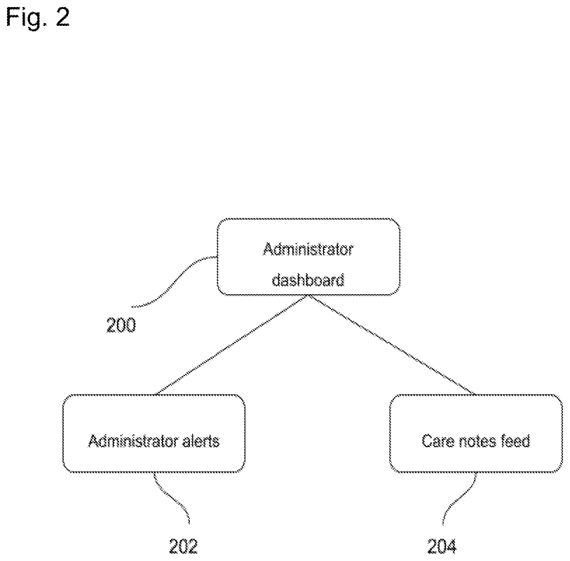

Referring to FIG. 2, the administrator dashboard module 200 provides all the relevant information for administrators to review/address prioritized by urgency and/or compliance risk.

The dashboard module 200 contains two sections, administrator alerts 202 and care notes feed 204. The notes include any expired documents will be marked as urgent.

### Resident Module

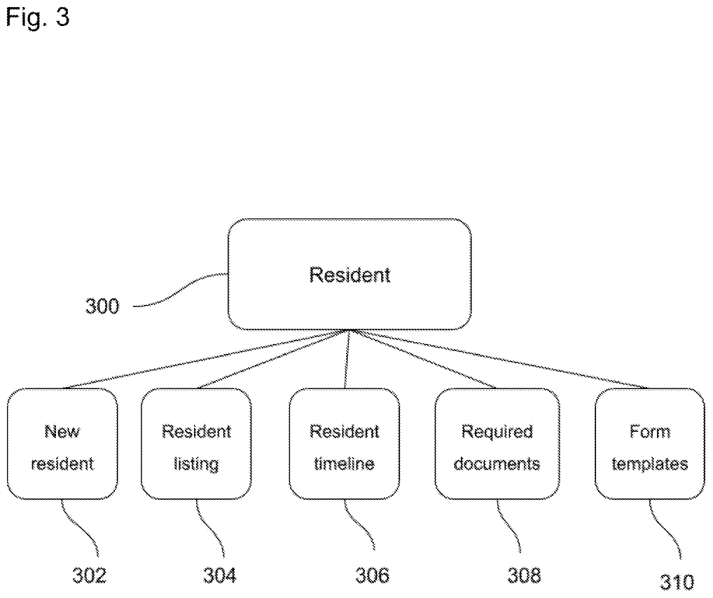

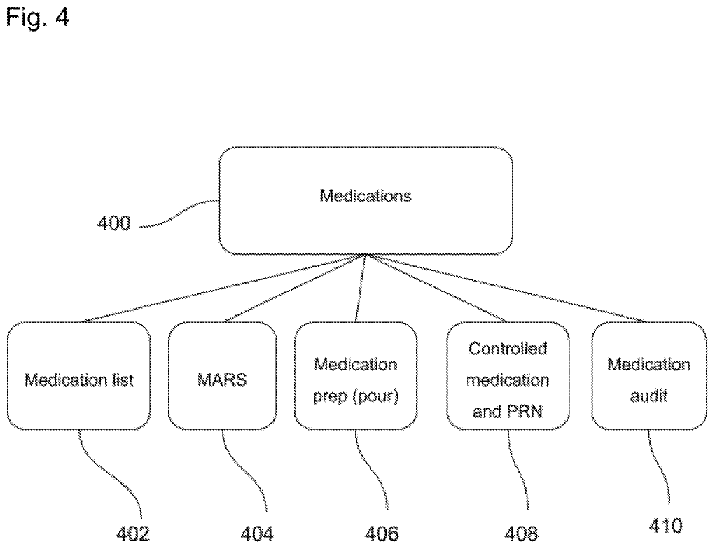

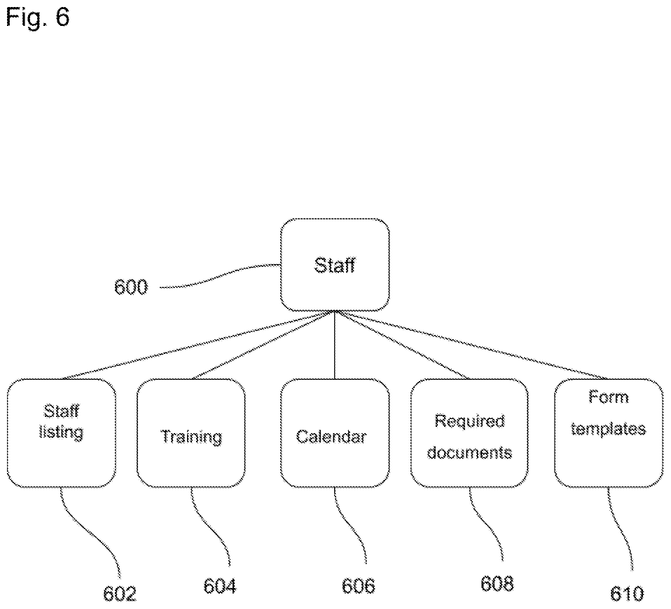

Referring to FIG. 3, the resident module 300 allows the care home to enter and maintain records for each of their residents 300 in a central location. This information is then available throughout the rest of the system and is also conveniently located for the staff 600 as shown in FIG. 6 to find in the event of an emergency. This module also provides medical personnel to enter relevant information regarding the resident's care into the system that will then be fed into the other relevant modules of the website like the medication module 400 as shown in FIG. 4. As residents 300 move in and move out, electronic records will be maintained for audit purposes.

The resident module 300 contains five sections, new resident 302, resident listing 304, resident timeline 306, required documents 308 and form templates 310 as following:

### New Resident

The new resident section 302 is to enter all new resident information, this section contains four tabs, Identification and info (LIC 601), Appraisal (LIC 603a), Physician report, and accommodation and fees.

### Resident Listing

The resident listing section 304 list any resident after they have been entered in the new resident section, in this section a user can click the residents name and it will takes him back to the above page so he can alter resident information, the resident listing section 304 also shows a birth date, sex and the admission date.

### Resident Timeline

The resident timeline section 306 is a feed of any caregiver portal entry ADLs 502, medication 506, admin 800, care note 504 and treatment.

### Required Documents

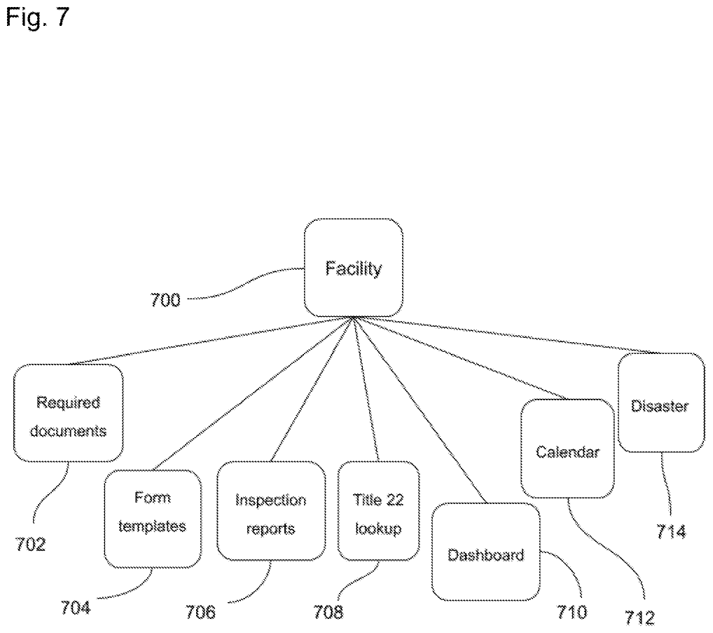

The required documents section 308 is where a user uploads and view required documents listed on California state form LIC 311 for each facility 700 as shown in FIG. 7. Here the user will upload each required document 308 for each resident 300, it can be viewed or upload. As upload user will enter an expiration date as well as a signature date, when completed a green checkmark will show under the word present to show compliance. This is an easy to view screen all residence and what is present and what is missing. This page contains resident name, whether the item is required, whether ifs present, and what action to take including view upload and print.

The documents and templates in the sections of required documents 308 and form templates 310 are:

| | | |
|---|---|---|
| 1. | LIC 603 | Resident Appraisal ARF |
| 2. | LIC 603A | Resident Appraisal RCFE |
| 3. | LIC 625 | Appraisal Need and Services Plan |
| 4. | LIC 602A | Physician's Report for RCFE |
| 5. | LIC 602 | Physician's Report for ARF |
| 6. | LIC 601 | Identification and Emergency Information |
| 7. | LIC 604A | Admission Agreement |
| 8. | LIC 613C | Personal Rights Information |
| 9. | LIC 405 | Residents Safeguarded Cash Resources |
| 10. | LIC 621 | Resident Personal Property and Valuables |
| 11. | LIC 624 | Unusual Incident/lnjury Report |
| 12. | LIC 624A | Death Report |
| 13. | LIC 622 | Centrally Stored Medication and Destruction Record |
| 14. | LIC 9060 | Resident Theft and Loss Record |

### Medication Module

All of the medications 400 in the facility 700 as shown in FIG. 7 are to be entered in the Medications module 400. Each medication 400 is associated with the resident 300 who will be taking the medication 400 as well all the relevant information (dosage quantity and frequency, who prepared/poured the medications 400, who passed the medications 400 to the resident 300, etc.). The medication module 400 also allows tracking the medications 400 if the resident 300 leaves the facility 700 or if the medication 400 is destroyed (in the event of death). There is in depth reporting to indicate when medication 400 was prepared but never passed to the resident 300, there is alerts built in if the resident's medication 400 is nearly empty and refill needs to be ordered, Built in reporting allows the care home administrator 200 as shown in FIG. 2 to monitor the controlled medications 400 to ensure that they are not being mishandled or stolen. The system also restricts the care home staff 600 from pouring too much medication 400 or passing the medication 400 too frequently. There are also reminders built in if a dose was missed or if the resident 300 refused to accept the medication 400.

The medication module 400 contains five sections, medication list 402, MARS 404, medication prep (pour) 406, controlled medication and PRN 408, and medication audit 410 as follows:

### Medication List:

In the medication list section 402, a user enters a new drug and the required information, then it is automatically transferred to the mars 404 and stored meds LIC622 form. Here user can see an entire list of medications 400 in the facility 700 or break it down per resident 300 this is also where a user add medication 400 when clicking add medication screen comes up and collects all data on every prescription. This includes resident name, prescription type, medication type, dosage frequency, dosage strength, quantity, unit, supply quantity, instructions, expiration date, date filled, date started, and date, prescribing physician, pharmacy name, prescription number, number of refills, and refill expiration you can also check the box controlled medication or PRN medication 408.

### MARS:

In the MARS section 404, MARS stands for medication administration record sheet, there are thirty one boxes that go across and there is a spot for each medication 400 to be listed each thirty one box represents a date the medication 400 was given the signatures will go from the caregiver med pass to the MARS sheet eat 404. On the screen the user will choose the resident 300 and the month these MARS 404 available for reprint anytime.

### Medication Prep (Pour):

In the section of medication prep (pour) 406 (Prep means to prepare medications 400. Pour is to pour from a bottle or remove a tablet from a bottle. Some facilities say to “prepare medications” and some say “Pour medications” they are both the same essentially). User reads screen and sets up doses of medications 400, checks that they are set up. This information goes to caregiver portal medication 506 pass.

### Controlled Medications and PRN:

In the section of controlled medications and PRN 408, when a user click here he will be brought to either his controlled medication sheet or PRN sheet with the resident 300 drop-down menu on top

### Medication Audit:

The Medication audit section 410 will contain any medication errors missed dosage per resident 300.

### Caregiver Portal Module:

The caregivers module 500 provides a single focused tool for caregivers 500 to perform their duties and to have everything documented in the system. This not only provides a record of the activities for future reference, but it also provides reminders and alerts when appropriate (i.e. a reminder to check on a resident in two hours after giving them pain medication 506 to see if the medication 506 was effective).

This module is designed to be a one-stop-shop for caregivers 500. These screens give them access to track ADL's 502, provide care notes 504 throughout the day, pass medications 506 to the residents 300 (as shown in FIG. 3), and it also provides a timeline 508 which summarizes the residents' care over time. The timeline 508 also provides a way for a care home owner or administrator to monitor the overall well being of the care home's residents.

In the Caregiver 500 module, a user have four sections, ADL's 502, care notes 504, medication 506, timeline 508 and resident profile 510 as follows.

### ADLs:

The ADL's section 502 will have a full list of all. ADLs 502 including self-administration of meds, treatments, eating, escort to meals, snacks, room tray, dressing, clothing choice, change depends/diapers, perry wash, sponge bath, full shower, oral care, hair care, transferring, toileting assistance, urine, bowel movement, recreational activities, escort activity, appointments, change sheets, housekeeping, laundry, tidy room, dentures, hearing aid karma glasses, wait, blood pressure, blood sugar, INR Coumadin level, outside care or visitor, passive supervision, safety check, walking, social services, transportation. The next column has all drop down menus to select independent, refused or staff assist. The next row is an area to write notes and above that all is where a user selects his resident.

### Care Notes:

In the care notes section 504 there is a select resident button care note upload file and a checkbox for submit administrator review if this box is checked in immediately goes to the administrator's dashboard 200 with an alert 202 as shown in FIG. 2.

### Medication

In the medication section 506 a user will click the resident 300 and it will automatically generate the medications that are beat to be passed at what our what the name of them are and have a checkbox for the caregiver 500 to check past or refused there is a note section and the unique dispenser code.

### Timeline:

The timeline section 508 summarizes the residents' care over time. The timeline 508 also provides a way for a care home owner or administrator to monitor the overall well being of the care home's residents.

### Resident Profile:

The resident profile section 510 collects data from intake or new resident section 302 as shown in FIG. 3 photo and several other places to create easy to use resident profile sheets for staff.

### Staff Module:

The staff module 600 provides a central location for entering and referencing the staff contact information, training documentation, and provides scheduling and basic HR information.

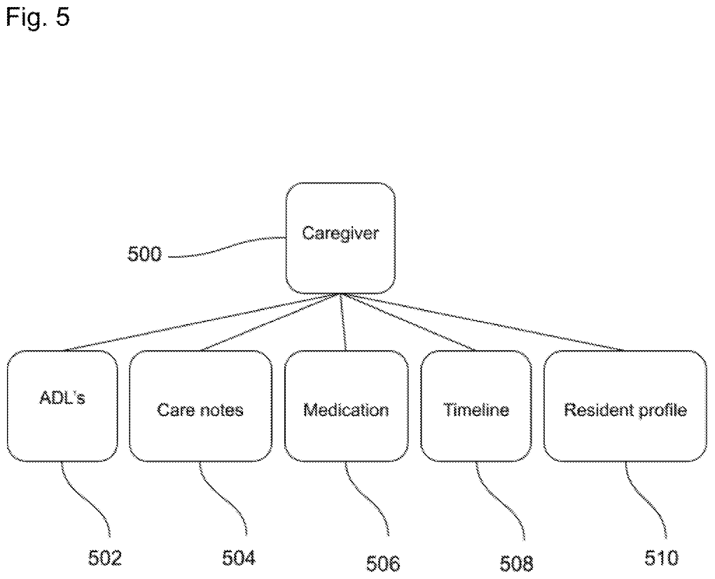

Each of the care home staff members 600 are setup with a profile and are granted specific access by their administrator. If they are a caregiver 500 as shown in FIG. 5, they will just have access to the caregiver module 500 as shown in FIG. 5 which allows them access to perform their day-to-day functions but does not allow them access to other staff members data or private resident data (i.e. SSN). A staff member 600 setup as an administrator will have full access to all the application at the care home level.

The staff module 600 also provides a place to track all the staff members' training 604 to ensure compliance with the training requirements.

The staff module 600 contains five sections, staff listing 602, training 604, calendar 606, required documents 608, and form templates 610 as follows:

### Staff Listing:

In the staff listing 602 there is a button that says add staff, when the user press add staff, the user will see a List of all staff members.

### Training:

In the training section 604, the user clicks add training and fill out a training log that includes staff member, training types, training title, training description, completion date, renewal date, it also has an upload button.

### Calendar:

In the calendar section 606, the user will find a calendar for activities, meds, doctor appointments, safety checks and night changes meetings.

### Required Documents:

The required documents section 608 contains the staff required documents 608 they are the same as resident required documents 308 as shown in FIG. 3.

### Staff Required Documents:

| 1. | Health Screening Report/Tuberculosis (TB) Clearance - Facility Staff | |
|---|---|---|
| 2. | Tuberculosis (TB) Clearance and “Good Health” | |
| | Statement - Volunteers | |
| 3. | Reports of Actual Hours Worked by Staff | |
| 4. | LIC 501 | Personnel Record |
| 5. | Age Verification/Copy of Current ID | |
| 6. | Administrator Certificate | |
| 7. | Documentation of Annual 20 clock-hour Training | |
| 8. | LIC 508 | Criminal Record Statement |

| | |
|---|---|
| 9. | Criminal Record Clearance or Exemption |
| 10. | Verification of Staff Training |
| 11. | Verification of CPR Training |
| 12. | Verification of First Aid Training |
| 13. | Staff Credentials/Certifications |

### Form Templates:

In the form templates section 610 there is the LIC501 form, the user will fill out the staffs first name, last name, birthdate, phone number, email address, address line, city, zip, you will assign a user ID, a sign password, confirm password, last physical exam, last TB test, date of hire, signature date, and the user will have the option to choose the form lic501.

### Facility Module:

The facility module 700 allows user to setup and maintain all the information regarding their facility 700 in the system as well as upload all the required documentation into one location. The facilities information is used for automatically determining compliance based on the classification, number of beds, type of patients (i.e. dementia, bedridden, etc.).

The facility module 700 contains 7 sections, required documents 702, form templates 704, inspection reports 706, title 22 lookup 708, dashboard 710, Calendar 712 and disaster 714 as follows:

The required documents section 702 include:

| 1. | RCFE License | |
|---|---|---|
| 2. | LIC 500 | Personnel Report |
| 3. | Verification of First Aid Training for Staff Providing Care | |
| 4. | Written First Aid Procedures | |
| 5. | Menus (16+ Residents: Min 30 Days History; <16 Residents: Sample Menu) | |
| 6. | LIC 610E | Emergency Disaster Plan |
| 7. | LIC 401 | Financial Records Including Income and Expenditures |
| 8. | LIC 402 | Surety Bond |
| 9. | Plan of Operation: Plan for Staff Trainings | |
| 10. | Plan of Operation: Facility Sketches | |
| 11. | Plan of Operation: Statement RE: Handling Client Monies | |
| 12. | Plan of Operation: Plan for Hazardous Behaviors | |
| 13. | Plan of Operation: Policy for Facility Visits | |
| 14. | Neighborhood Complaint Policy | |
| 15. | Subscription to Service for Changes in Requirements | |
| 16. | Insurance Statement | |
| 17. | Fire Inspection Report | |
| 18. | 16+ Residents: Dated Weekly Employee Time Schedule | |
| 19. | Licensee Affidavit on Persons Exempt from Fingerprint Requirements | |
| 20. | Appropriate Driver's License for Each Staff Member Transporting Residents | |
| 21. | 7+ Residents: Notices of Planned Activities (Min 6 Months History) | |
| 22. | 50+ Residents: Program of Activities | |
| 23. | 50+ Residents: Evidence of Consultation from Nutritionist, Dietitian, or Home | |
| | Economist | |
| 24. | LIC 610 | Emergency Disaster Plan |
| 25. | LIC 215 | Applicant Information |
| 26. | LIC 200 | Application for Community Care Facility |
| 27. | LIC 9092 | Fire Pre-Inspection/Consultation Request |
| 28. | LIC 309 | Administrative Organization |
| 29. | LIC 198 | Child Abuse Central Index |
| 30. | LIC 400 | Affidavit Regarding Resident Cash Resources |
| 31. | LIC 308 | Designation of Facility Responsibility |
| 32. | LIC 9054 | Local Fire Inspection Authority Information |
| 33. | LIC 9020 | Register Of Facility Clients/Residents |
| 34. | LIC 401a | Supplemental Financial Information |
| 35. | LIC 403 | Balance Sheet |
| 36. | LIC 404 | Financial Information Release And Verification |
| 37. | LIC 999 | Facility Sketch (Floor Plan) |

| | |
|---|---|
| 38. | Board of Directors Statement (if applicable) |
| 39. | Corporation Bylaws (if applicable) |
| 40. | Dementia Plan of Operation |
| 41. | Hospice Waiver Documentation |
| 42. | Theft and Loss Policy |
| 43. | Admission Agreement |

The form templates section 704 includes blank forms for use.

In the inspection report section 706 a user uploads inspection reports and dates when inspection was performed.

In the title 22 lookup section 708 (Title 22 Regulations of the California Code contains rules concerning community care facilities in the state.) Child Care and residential care facilities fall under the purview of the Community Care Licensing Division and must follow these regulations. Facilities for children, the chronically ill and the elderly, along with foster homes and social rehabilitation facilities, title 22 for licensing and operating regulations. Operating rules for each type of facility 700 include staffing requirements, staff-resident ratios and resident rights.). PDF of title 22 with the option to click the find box and type in subjects for title 22 regulation numbers.

### Dashboard:

In the dashboard section 710 there are administrator alerts, care notes feed under administrator alerts, all alerts regarding missing documents, medications becoming do, incident reports, anniversary dates and birthdays.

### Calendar:

In the calendar section 712, the user will find a calendar for activities meds doctor appointments safety checks night changes meetings. calendar's are for staff, resident and facility.

In the disaster section 714, comprehensive disaster planning and all information required for AB 3098 RCFE (AB 3098 RCFE stands for Assembly Bill No. 3098 Residential care facilities for the elderly, emergency and disaster plans.) disaster plan requirements including resident forms lic 603 and lie 622, facility forms lie 9020 and lic 500.

The disaster required documents are:

| 1. | LIC 500 | Personnel Report |
|---|---|---|
| 2. | LIC 610E | Emergency Disaster Plan |
| 3. | Plan of Operation: Facility Sketches | |
| 4. | Fire Inspection Report | |
| 5. | LIC 603A | Resident Appraisal RCFE |
| 6. | LIC 625 | Appraisal Need and Services Plan |
| 7. | LIC 601 | Identification and Emergency Information |
| 8. | LIC 622 | Centrally Stored Medication and Destruction Record |
| 9. | LIC 9060 | Resident Theft and Loss Record |

### Admin Module:

The admin module 800 contains two sections, users 802 and system settings 804 as follows?

### Users:

In the user section 802, a user will create user IDs for facility 700 (as shown in FIG. 7).

### System Settings:

The system settings section 804 is to collect all data and converts into a report.

### Licensed Program Analyst (LPA) Module:

The Licensed Program Analyst (LPA) module 900 provides a concise view of the facilities required documents allowing an LPA to quickly identify areas of the facilities documents that is potentially out of compliance and should be reviewed. The system and methods for ensuring facility compliance plans to build a compliance dashboard which will summarize several of the key metrics and activities of the facility inspection process to audit for compliance and/or for insurance companies to determine the overall risk and compliance of a facility. This compliance dashboard will allow the state to view all facilities. With this functionality, the state can prioritize which care homes to audit earlier based on overall risk. It would also be possible to assign LPA users to specific care homes and/or counties within the state that they are responsible for reviewing.

In the licensed program analyst (LPA) module 900 there is a user name and password given to LPA who can then log in and view only required documents section of the program. The LPA will have access to all facility 700 as shown in FIG. 7, Staff 600 as shown in FIG. 6 and resident required documents 308 as shown in FIG. 3.

Although the invention has been explained in relation to its preferred embodiment, it is to be understood that many other possible modifications and variations can be made without departing from the spirit and scope of the invention.

The foregoing descriptions of pre-defined embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed is:

1. An electronic record system and method to ensure facility compliance to operate a state licensed adult residential care facility comprising the modules of:
administrator dashboard;
resident;
medications;
caregivers;
staff;
facility;
admin; and,
licensed program analyst (LPA).

2. The administrator dashboard module of the electronic record system and method of claim 1 provides all the relevant information for administrators to review/address prioritized by urgency and/or compliance risk.

3. The administrator dashboard module of the electronic record system and method of claim 1 contains two sections, administrator alerts and care notes feed.

4. The resident module of the electronic record system and method of claim 1 allows the care home to enter and maintain records for each of their residents in a central location, this information is then available throughout the rest of the system and is also conveniently located for the staff to find in the event of an emergency.

5. The resident module of the electronic record system and method of claim 1 provides medical personnel to enter relevant information regarding the resident's care into the system that will then be fed into the other relevant modules of the website like the medication module, as residents move in and move out. electronic records will be maintained for audit purposes.

6. The resident module of the electronic record system and method of claim 1 contains five sections. new resident, resident listing, resident timeline, required documents and form templates.

7. The new resident section of the resident module of claim 6 is to enter all new resident information, this section contains four tabs, Identification and info (LIC 601), appraisal (LIC 603a), physician report, and accommodation and fees.

8. The resident listing section of the resident module of claim 6 list any resident after they have been entered in the new resident section, in the resident listing section a user can click the residents name and it will takes him back to the above page so he can alter resident information, the resident listing section shows a birth date, sex and the admission date.

9. The resident timeline of the resident module of claim 6 is a feed of any caregiver portal entry ADLs, medication, admin, care note and treatment.

10. The required documents section of the resident module of claim 6 is where a user uploads and view required documents listed on California state form LIC 311 for each facility, and the user will upload each required document for each resident, it can be viewed or upload, as upload user will enter an expiration date as well as a signature date, when completed a green checkmark will show under the word present to show compliance. this page contains resident name, whether the item is required, whether it's present, and what action to take including view upload and print.

11. The medications module of the electronic record system and method of claim 1 contains all of the medications in the facility, each medication is associated with the resident who will be taking the medication as well all the relevant information dosage quantity and frequency, who prepared and poured the medications, who passed the medications to the resident.

12. The medication module of the electronic record system and method of claim 1 allows tracking the medications if the resident leaves the facility or if the medication is destroyed in the event of death.

13. The medication module of the electronic record system and method of claim 1 wherein there is in depth reporting to indicate when medication was prepared but never passed to the resident.

14. The medication module of the electronic record system and method of claim 1 wherein there is alert built in if the resident's medication is nearly empty and refill needs to be ordered.

15. The medication module of the electronic record system and method of claim 1 wherein there is a built in reporting allows the care home administrator to monitor the controlled medications to ensure that they are not being mishandled or stolen.

16. The medication module of the electronic record system and method of claim 1 wherein the system also restricts the care home staff from pouring too much medication or passing the medication too frequently.

17. The medication module of the electronic record system and method of claim 1 wherein there are reminders built in if a dose was missed or if the resident refused to accept the medication.

18. The medication module of the electronic record system and method of claim 1 contains five sections, medication list, MARS, medication prep (pour), controlled medication and PRN, and medication audit.

19. The medication list section of the medication module of claim 18 wherein a user enters a new drug and the required information, then it is automatically transferred to the mars and stored meds LIC622 form where a user can see an entire list of medications in the facility or break it down per resident this is also where a user add medication when clicking add medication screen comes up and collects all data on every prescription, this includes resident name, prescription type, medication type, dosage frequency, dosage strength, quantity, unit, supply quantity, instructions, expiration date, date filled, date started, and date, prescribing physician, pharmacy name, prescription number, number of refills. and refill expiration you can also check the box controlled medication or PRN medication.

20. The MARS section of the medication module of claim 18 wherein MARS stands for medication administration record sheet, where there are thirty one boxes that go across and there is a spot for each medication to be listed each thirty one box represents a date the medication was given the signatures will go from the caregiver med pass to the MARS sheet eat, on the screen the user will choose the resident and the month these MARS available for reprint anytime.

21-72. (canceled)
